(19) United States
(12) Patent Application Publication
(10) Pub. No.: US 2022/0375074 A1
(43) Pub. Date: Nov. 24, 2022

(54) MEDICAL IMAGE PROCESSING APPARATUS

(71) Applicant: FUJIFILM CORPORATION, Tokyo (JP)

(72) Inventor: Kazuhiro MAKINO, Kanagawa (JP)

(21) Appl. No.: 17/742,408

(22) Filed: May 12, 2022

(30) Foreign Application Priority Data
May 18, 2021 (JP) 2021-083789

(51) Int. Cl. G06T 7/00

(52) U.S. Cl. CPC G06T 7/0012; G06T 2207/10081; G06T 2207/10116

(57) ABSTRACT

The medical image processing apparatus includes a first processor, a second processor that executes image processing on a medical image in response to an instruction from the first processor, and a battery that supplies power to the first processor and the second processor. After the second processor executes the image processing, transition is made to a power saving mode where an amount of power consumption in the second processor is relatively small.

### CROSS-REFERENCE TO RELATED APPLICATIONS

The present application claims priority under 35 U.S.C. § 119 to Japanese Patent Application No. 2021-083789, filed on May 18, 2021. The above application is hereby expressly incorporated by reference, in its entirety, into the present application.

### BACKGROUND

### 1. Technical Field

A technique of the disclosure relates to a medical image processing apparatus.

### 2. Description of the Related Art

As a technique regarding a medical image processing apparatus that processes a medical image, such as a radiographic image, the following technique is known. For example, JP2008-073121A describes a radiographic image forming system that monitors a use state of an X-ray source, a reading device, or a battery, and in a case where a state in which the X-ray source is not disposed at a position capable of performing imaging, a state in which there is no input of a signal to the reading device within a predetermined time, or a state in which a residual quantity of the battery is equal to or less than an allowable lower limit value is detected, sets the reading device in a waiting state or a power-off state in which power consumption is low.

JP2005-077905A describes a radiographic image reading device that has, as a mode at the time of non-operation, a mode where power supply to all parts is stopped and a waiting mode where power supply to a predetermined part is continued.

### SUMMARY

There is known a medical image processing apparatus that provides information useful for diagnosis, such as detecting and presenting a lesion from a medical image, by executing image processing of analyzing a medical image, such as a radiographic image, using a computer. Diagnosis support accompanied with the image processing using the computer is referred to as computer aided diagnosis (CAD). The CAD processing is accompanied with the image processing on the medical image. Thus, in a case where a processor specialized for image processing, such as a graphics processing unit (GPU), is made to execute the CAD processing, it is possible to considerably reduce a processing time compared to a case where a central processing unit (CPU) that is good at general-purpose processing.

On the other hand, it has been suggested that a CAD function is implemented in a mobile radiography apparatus (so-called treatment cart) comprising an irradiation unit that performs irradiation of radiation, a console, and a battery. The CAD function that is implemented in the mobile radiography apparatus is realized by the GPU independent of the console, whereby it is possible to promptly perform diagnosis support with the CAD function at a destination. Note that, in this case, power supply from the battery to the GPU is required and an amount of power supply from the battery increases. As a result, it is expected that the operation time of the apparatus is shortened or a replacement frequency of the battery increases, and efficient rounds may be obstructed.

The technique of the disclosure has been accomplished in view of the above-described points, and an object of the technique of the disclosure is to provide a medical image processing apparatus including a processor that executes image processing on a medical image, having an advantage of suppressing an amount of power consumption in the processor.

A medical image processing apparatus according to the technique of the disclosure comprises a first processor, a second processor that executes image processing on a medical image in response to an instruction from the first processor, and a battery that supplies power to the first processor and the second processor. After the second processor executes the image processing, transition is made to a power saving mode where an amount of power consumption in the second processor is relatively small.

The second processor may operate in synchronization with a clock signal of a relatively long cycle in the power saving mode. The second processor may perform communication with the first processor at a relatively low frequency in the power saving mode. The second processor may transition to a sleep state defined in advance, in the power saving mode. In the power saving mode, supply of power from the battery to the second processor may be cut off.

The first processor may transmit a release instruction of the power saving mode to the second processor in a case where a predetermined processing stage among a plurality of processing stages until the image processing is executed is performed. The first processor may transmit a release instruction of the power saving mode to the second processor in a case where an execution instruction of the image processing is received. The first processor may transmit a release instruction of the power saving mode to the second processor in a case where the release instruction of the power saving mode is received. The first processor may determine a timing of transmitting a release instruction of the power saving mode such that the second processor is returned to a state in which the image processing is possible, until a time of acquisition of the medical image.

The medical image may be a radiographic image. In this case, the medical image processing apparatus may further comprise a radiation irradiation unit that receives the supply of power from the battery to perform irradiation of radiation for capturing the radiographic image. The second processor may output information for supporting diagnosis using the medical image through the image processing. In the medical image processing apparatus, a first battery that supplies power to the first processor, and a second battery that supplies power to the second processor may be provided. The medical image processing apparatus may be a mobile type.

According to the technique of the disclosure, it is possible to provide a medical image processing apparatus comprising a processor that executes image processing on a medical image, having an advantage capable of suppressing an amount of power consumption in the processor.

### BRIEF DESCRIPTION OF THE DRAWINGS

Exemplary embodiments according to the technique of the present disclosure will be described in detail based on the following figures, wherein:

FIG. 1 is a block diagram showing an example of the configuration of a medical examination system according to an embodiment of the technique of the disclosure;

FIG. 2 is a side view showing an example of the appearance of the medical image processing apparatus according to the embodiment of the technique of the disclosure;

FIG. 3 is a perspective view showing an example of a capturing method of a radiographic image;

FIG. 4 is a block diagram showing an example of the configuration of a radiation irradiation unit according to the embodiment of the technique of the disclosure;

FIG. 5 is a diagram showing an example of the hardware configuration of a console according to the embodiment of the technique of the disclosure;

FIG. 6 is a diagram showing an example of the hardware configuration of a diagnosis support unit according to the embodiment of the technique of the disclosure;

FIG. 7 is a diagram showing an example of processing that is executed in a learning phase where a detection model according to the embodiment of the technique of the disclosure is made to perform learning through machine learning;

FIG. 8 is a flowchart illustrating an example of a flow of medical examination processing according to the embodiment of the technique of the disclosure;

FIG. 9 is a functional block diagram showing an example of the functional configuration of the console according to the embodiment of the technique of the disclosure;

FIG. 10 is a flowchart illustrating a flow of processing that is executed by executing a mode switching program according to the embodiment of the technique of the disclosure;

FIG. 11 is a functional block diagram showing an example of the functional configuration of the diagnosis support unit according to the embodiment of the technique of the disclosure;

FIG. 12 is a flowchart illustrating a flow of processing that is executed by executing a CAD processing program according to the embodiment of the technique of the disclosure; and

FIG. 13 is a block diagram showing an example of the configuration of the medical image processing apparatus according to the embodiment of the technique of the disclosure.

### DETAILED DESCRIPTION

Hereinafter, an example of an embodiment of the technique of the disclosure will be described referring to the drawings. In the drawings, the same or equivalent components are represented by the same reference numerals, and overlapping description will not be repeated.

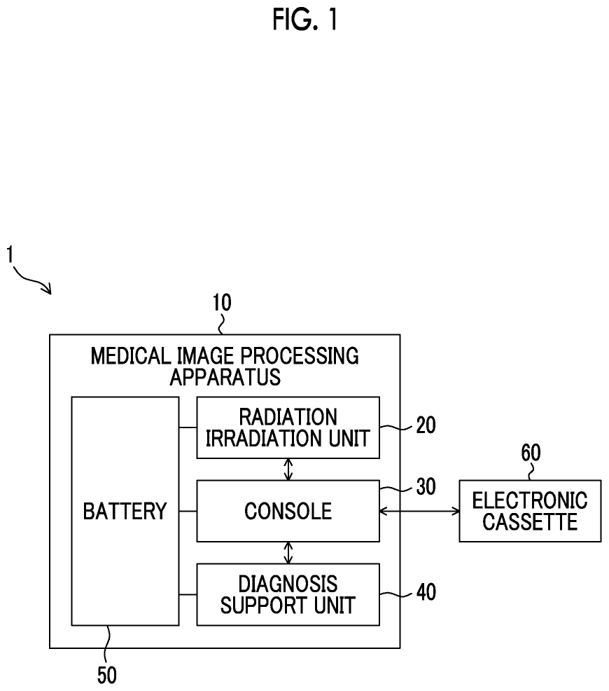

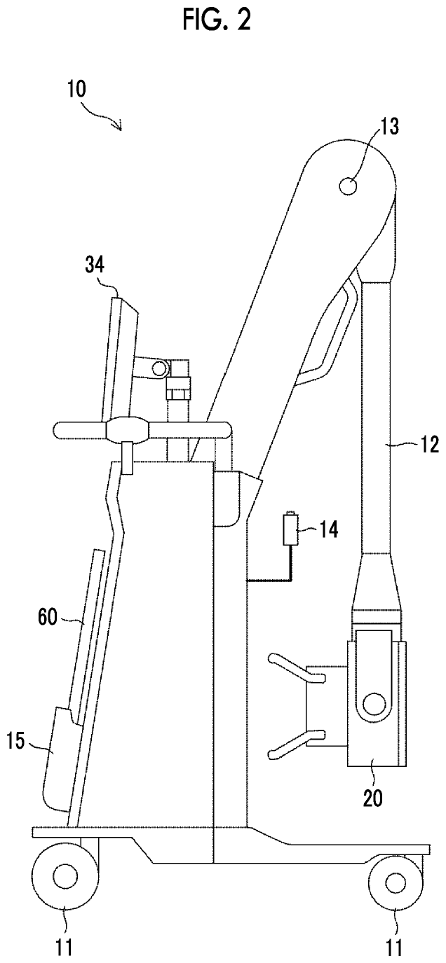

FIG. 1 is a diagram showing an example of the configuration of a medical examination system 1 according to an embodiment of the technique of the disclosure. The medical examination system 1 includes a medical image processing apparatus 10 and an electronic cassette 60. FIG. 2 is a side view showing an example of the appearance of the medical image processing apparatus 10. The medical image processing apparatus 10 has a function of acquiring a radiographic image that is obtained by irradiating a patient as a subject with radiation, such as X-rays, executing CAD processing accompanied with image processing on the radiographic image, and presenting a result of the CAD processing. The radiographic image is an example of a “medical image” in the technique of the disclosure and is generated by an electronic cassette 60.

As shown in FIG. 2, the medical image processing apparatus 10 has wheels 11 in a bottom portion. That is, the medical image processing apparatus 10 is a portable mobile type. Accordingly, the medical image processing apparatus 10 can be used for rounds in which a physician goes round and examines inpatients in a hospital ward. As shown in FIG. 1, the medical image processing apparatus 10 comprises a radiation irradiation unit 20, a console 30, a diagnosis support unit 40, and a battery 50.

The radiation irradiation unit 20 has a function of performing irradiation of radiation, such as X-rays, with which the subject is irradiated, in a case of capturing a radiographic image. The radiation irradiation unit 20 is provided at a distal end of an arm part 12. The arm part 12 can expand and contract in a longitudinal direction and can rotate with a shaft part 13 as a rotation axis.

The console 30 and the diagnosis support unit 40 include computers independent of each other. The battery 50 supplies power to each of the radiation irradiation unit 20, the console 30, and the diagnosis support unit 40. The battery 50 is a secondary battery, such as a lithium polymer battery and can be charged through a connector (not shown). The console 30, the diagnosis support unit 40, and the battery 50 are incorporated in the medical image processing apparatus 10.

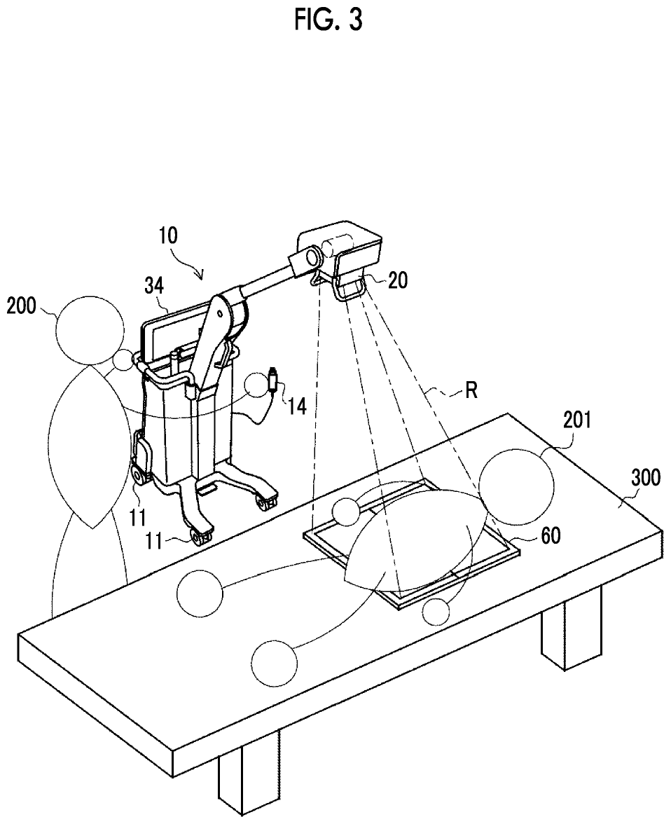

FIG. 3 is a perspective view showing an example of a method of capturing a radiographic image using the medical image processing apparatus 10 and the electronic cassette 60. FIG. 3 illustrates a case of capturing a radiographic image of a chest of a subject 201 in a supine state on an examination table 300. The electronic cassette 60 is disposed at a position facing the radiation irradiation unit 20. The subject 201 is disposed between the radiation irradiation unit 20 and the electronic cassette 60 such that an imaging target part falls within an irradiation field of radiation.

A user 200, such as a radiology technician or a physician, operates an irradiation switch 14, whereby irradiation of radiation R is performed from the radiation irradiation unit 20. The radiation R transmitted through the subject 201 reaches the electronic cassette 60. The electronic cassette 60 is a known portable flat panel detector (FPD) that detects the radiation R transmitted through the subject 201 to generate a radiographic image. The electronic cassette 60 has a function of automatically detecting an irradiation start of the radiation R from the radiation irradiation unit 20. For this reason, the electronic cassette 60 can generate a radiographic image without being connected to the medical image processing apparatus 10. The electronic cassette 60 has a wireless communication function and transmits the generated radiographic image to the console 30 through wireless communication. The medical image processing apparatus 10 has a housing portion 15 (see FIG. 2) that houses the electronic cassette 60. In a state in which the electronic cassette 60 is housed in the housing portion 15, a battery (not shown) incorporated in the electronic cassette 60 can be charged.

Hereinafter, each constituent element of the medical image processing apparatus 10 shown in FIG. 1 will be described in detail.

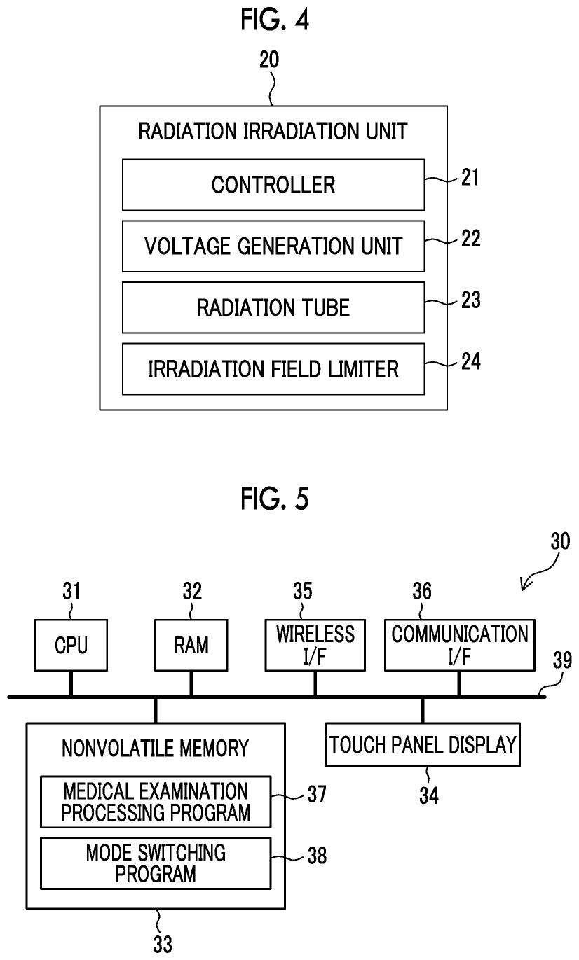

FIG. 4 is a block diagram showing an example of the configuration of the radiation irradiation unit 20. The radiation irradiation unit 20 comprises a controller 21, a voltage generation unit 22, a radiation tube 23, and an irradiation field limiter 24. The radiation tube 23 includes a filament, a target, and a grid electrode (all are not shown). A voltage that is output from the voltage generation unit 22 is applied across the filament as a cathode and the target as an anode. The voltage that is applied across the filament and the target is referred to as a tube voltage. The filament emits thermoelectrons depending on the applied tube voltage toward the target. The target emits radiation with collision of thermoelectrons from the filament. The grid electrode is disposed between the filament and the target. The grid electrode controls a flow rate of the thermoelectrons from the filament toward the target. The flow rate of the thermoelectrons from the filament toward the target is referred to as a tube current. The controller 21 controls the tube voltage, the tube current, and an irradiation time of radiation based on an instruction from the console 30.

The irradiation switch 14 is a two-stage push type switch that is provided for the user, such as a radiology technician or a physician, to give an instruction to start the irradiation of the radiation. In a case where the irradiation switch 14 is pushed to a first stage, the filament is warmed up, and the rotation of the target is started. When the filament reaches a prescribed temperature, and the target reaches a prescribed rotation speed, warm-up is completed. In a state in which warm-up is completed, in a case where the irradiation switch 14 is pushed to a second stage, the voltage is applied from the voltage generation unit 22, and radiation is emitted from the radiation tube 23.

The irradiation field limiter 24 limits an irradiation field of the radiation emitted from the radiation tube 23. The irradiation field limiter 24 has, for example, a configuration in which four shield plates that shield the radiation are disposed on respective sides of a quadrangle, and an opening of the quadrangle that transmits the radiation is formed in a center portion. The irradiation field limiter 24 changes the positions of the four shield plates to change the size of the opening, and accordingly, changes the size of the irradiation field of the radiation.

The console 30 is a computer that integrally controls various kinds of processing to be executed in the medical image processing apparatus 10. FIG. 5 is a diagram showing an example of the hardware configuration of the console 30. The console 30 has a CPU 31, a random access memory (RAM) 32, a nonvolatile memory 33, a touch panel display 34, a wireless interface 35, and a communication interface 36. The CPU 31, the RAM 32, the nonvolatile memory 33, the touch panel display 34, the wireless interface 35, and the communication interface 36 are connected to a bus 39.

The nonvolatile memory 33 is a storage device, such as a flash memory, and stores a medical examination processing program 37 and a mode switching program 38 described below. The RAM 32 is a work memory on which the CPU 31 executes processing. The CPU 31 loads each program stored in the nonvolatile memory 33 to the RAM 32 and executes processing depending on each program. The CPU 31 is an example of a “first processor” in the technique of the disclosure.

The touch panel display 34 functions as an input device that receives an input of information for processing to be executed by the CPU 31 and an output device that outputs a result of processing executed by the CPU 31. The input device may include known input means, such as operation buttons, a hardware keyboard, a mouse, and a track ball.

The wireless interface 35 is an interface that is provided for the console 30 to perform transmission and reception of information or data through wireless communication with the electronic cassette 60 and other equipment. The console 30 acquires a radiographic image that is transmitted from the electronic cassette 60 through wireless communication, through the wireless interface 35. The acquired radiographic image is stored in the nonvolatile memory 33.

The communication interface 36 is an interface that is provided for the console 30 to perform transmission and reception of information or data with the diagnosis support unit 40 and other equipment. The communication interface 36 may be, for example, a communication interface conforming to a universal serial bus (USB).

The diagnosis support unit 40 is a computer that executes CAD processing accompanied with image processing on a radiographic image in response to an instruction from the console 30. The diagnosis support unit 40 outputs information for supporting diagnosis using a medical image as a result of the CAD processing. The diagnosis support unit 40 detects an abnormal shadow, such as a lesion part, included in the radiographic image as the CAD processing and transmits a result of the detection to the console 30. The diagnosis support unit 40 is configured of a computer independent of the console 30.

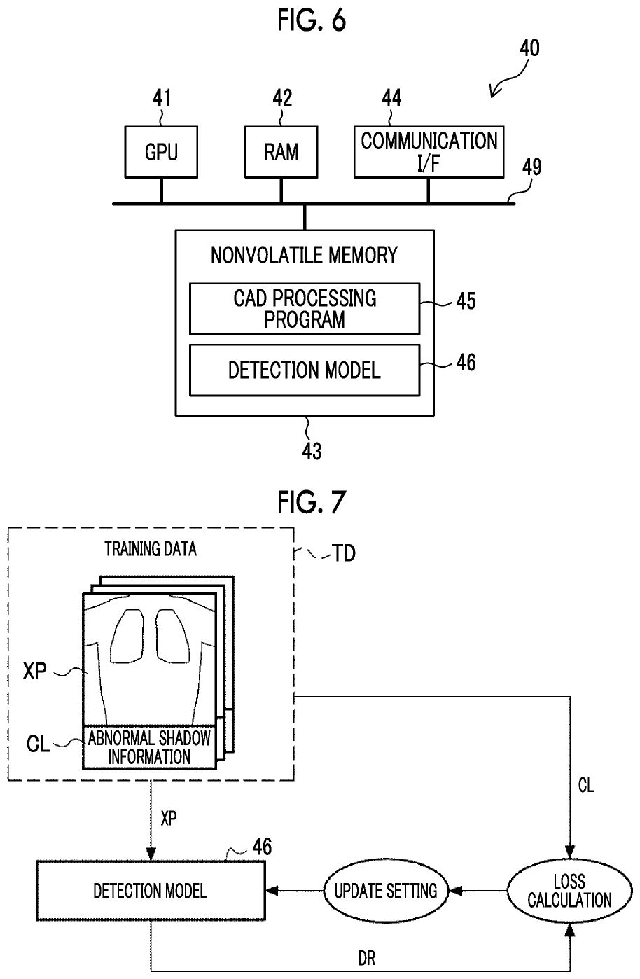

FIG. 6 is a diagram showing an example of the hardware configuration of the diagnosis support unit 40. The diagnosis support unit 40 has a graphic processing unit (GPU) 41, a RAM 42, a nonvolatile memory 43, and a communication interface 44. The GPU 41, the RAM 42, the nonvolatile memory 43, and the communication interface 44 are connected to a bus 49.

The GPU 41 is a processor that has a greater number of cores than the CPU 31 in the console 30 and can perform comparatively simple calculations, such as matrix operations, in parallel. For this reason, the GPU 41 can perform the CAD processing accompanied with the image processing of the radiographic image at a higher speed that the CPU 31. The GPU 41 is an example of a “second processor” in the technique of the disclosure.

The nonvolatile memory 43 is a storage device, such as a flash memory, and stores a CAD processing program 45 and a detection model 46 described below. The RAM 42 is a work memory on which the GPU 41 executes processing. The GPU 41 loads the CAD processing program 45 stored in the nonvolatile memory 43 to the RAM 42 and executes the CAD processing depending on the CAD processing program 45. The communication interface 44 is an interface that is provided for performing transmission and reception of information or data with the console 30 and other equipment. The communication interface 44 may be, for example, a communication interface conforming to a USB.

The diagnosis support unit 40 may have an attachable and detachable form of a so-called “external GPU box” comprising a housing that houses the GPU 41, the RAM 42, the nonvolatile memory 43, and the communication interface 44. The diagnosis support unit 40 may further comprise a CPU for general-purpose processing, in addition to the GPU 41. In this case, it is preferable that the GPU 41 professionally executes the image processing of the radiographic image, and the CPU executes general-purpose processing, such as execution control of a program and communication control with the console 30.

The detection model 46 is a mathematical model for detecting an abnormal shadow, such as a lesion part, included in the radiographic image and is a learned model that performs learning through machine learning. The detection model 46 is configured using, for example, a neural network. The detection model 46 is configured using, for example, a deep neural network (DNN) that is a multilayered neural network to be a target of deep learning. As the DNN, for example, a convolutional neural network (CNN) suitable for an image is used. As the radiographic image as a CAD processing target is input to the detection model 46, a detection result of an abnormal shadow, such as a lesion part, included in the radiographic image as a CAD processing target is output from the detection model 46.

FIG. 7 is a diagram showing an example of processing that is executed in a learning phase where the detection model 46 is made to perform learning through machine learning. The detection model 46 performs learning using training data TD. The training data TD includes a plurality of radiographic images XP attached with a correct answer label CL. The radiographic images XP included in the training data TD are sample images including various abnormal shadows. The correct answer label CL is, for example, positional information of an abnormal shadow in the radiographic image XP.

In the learning phase, the radiographic image XP is input to the detection model 46. The detection model 46 outputs a detection result DR that is a result of detecting an abnormal shadow from the input radiographic image XP. Loss calculation using a loss function is performed based on the detection result DR and the correct answer label CL. Then, update setting of various coefficients (weight coefficient, bias, and the like) of the detection model 46 is performed depending on a result of the loss calculation, and the detection model 46 is updated depending on the update setting.

In the learning phase, the series of processing of the input of the radiographic image XP to the detection model 46, the output of the detection result DR from the detection model 46, the loss calculation, the update setting, and the update of the detection model 46 is repeatedly executed. The repetition of the series of processing ends in a case where the detection accuracy of an abnormal shadow reaches a predetermined set level. The detection model 46 in which the detection accuracy reaches the set level is stored as a learned detection model in the nonvolatile memory 43. The detection model 46 is used for the CAD processing that is executed in the diagnosis support unit 40.

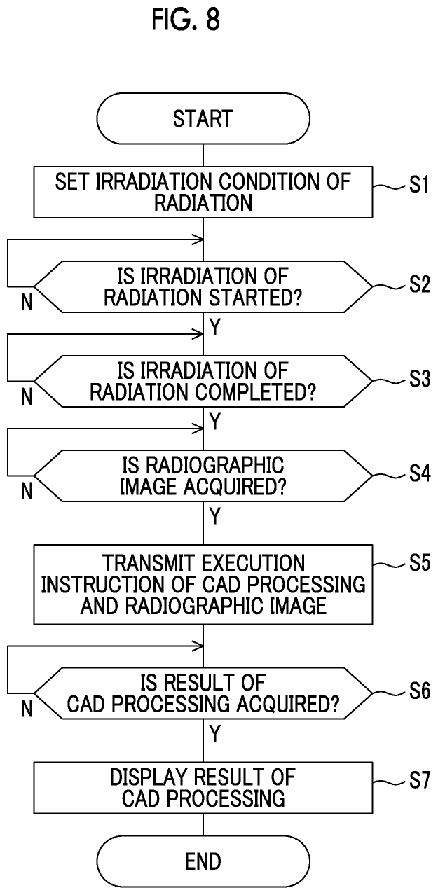

FIG. 8 is a flowchart illustrating an example of a flow of medical examination processing that is executed by the CPU 31 of the console 30 executing the medical examination processing program 37. The medical examination processing program 37 is executed, for example, in a case where the user, such as a radiology technician or a physician, gives an instruction to start the medical examination processing by operating the touch panel display 34.

In Step S1, the CPU 31 executes processing of setting irradiation conditions of radiation from the radiation irradiation unit 20. Specifically, the CPU 31 displays a selection screen of an imaging menu on the touch panel display 34 and receives a selection instruction of an imaging menu. The user, such as a radiology technician or a physician, selects an imaging menu corresponding to an imaging procedure designated in a medical examination order supplied from a radiology information system (RIS) (not shown). The console 30 can be connected to the RIS through the wireless interface 35. The CPU 31 supplies the irradiation conditions of the radiation including a tube voltage, a tube current, and an irradiation time corresponding to the selected imaging menu to the controller 21 of the radiation irradiation unit 20. With this, in the radiation irradiation unit 20, the irradiation conditions of the radiation including the tube voltage, the tube current, and the irradiation time are set. The user can correct the irradiation conditions of the radiation correlated with the imaging menu by operating the touch panel display 34.

In Step S2, the CPU 31 determines whether or not the irradiation of the radiation is started. The CPU 31 determines that the irradiation of the radiation is started, for example, in a case where detection is made that the irradiation switch 14 is pushed to the second stage.

In Step S3, the CPU 31 determines whether or not the irradiation of the radiation is completed. The CPU 31 determines that the irradiation of the radiation is completed, for example, in a case where determination is made that the irradiation time set in Step S1 has elapsed from a time at which the irradiation of the radiation is started.

The radiation that is emitted from the radiation irradiation unit 20 and is transmitted through the subject reaches the electronic cassette 60. The electronic cassette 60 detects the radiation transmitted through the subject to generate a radiographic image and transmits the generated radiographic image to the console 30 through wireless communication.

In Step S4, the CPU 31 determines whether or not the radiographic image transmitted from the electronic cassette 60 is acquired. In a case where determination is made that the radiographic image is acquired, the CPU 31 stores the acquired radiographic image in the nonvolatile memory 33 and transitions the process to Step S5.

In Step S5, the CPU 31 transmits an execution instruction of the CAD processing accompanied with the image processing on the acquired radiographic image to the diagnosis support unit 40 along with the radiographic image as a CAD processing target. The CPU 31 may transmit the execution instruction of the CAD processing and the radiographic image as a CAD processing target to the diagnosis support unit 40 in response to an instruction from the user.

In a case where the execution instruction of the CAD processing and the radiographic image as a CAD processing target are received, the diagnosis support unit 40 executes the CAD processing on the radiographic image as a CAD processing target and transmits a result of the CAD processing to the console 30.

In Step S6, the CPU 31 determines whether or not the result of the CAD processing transmitted from the diagnosis support unit 40 is acquired.

In Step S7, the CPU 31 displays the result of the CAD processing acquired in Step S6 on the touch panel display 34.

In this way, the medical image processing apparatus 10 according to the embodiment has a function of executing the CAD processing accompanied with the image processing on the acquired radiographic image, in addition to a function of capturing the radiographic image. Note that there is a need for power supply from the battery 50 even to the diagnosis support unit 40 including the GPU 41 that executes the CAD processing, and a supply amount of power from the battery 50 increases compared to a case where a CAD processing function is not provided. As a result, it is expected that an operation time of the medical image processing apparatus 10 is shortened or a replacement frequency of the battery 50 increases, and efficient rounds may be obstructed.

Accordingly, in the medical image processing apparatus 10 according to the embodiment, the diagnosis support unit 40 performs transition to a power saving mode and release of the power saving mode at a predetermined timing in response to an instruction from the console 30, thereby suppressing an amount of power consumption in the diagnosis support unit 40 (in particular, the GPU 41). Details of the power saving mode will be described below.

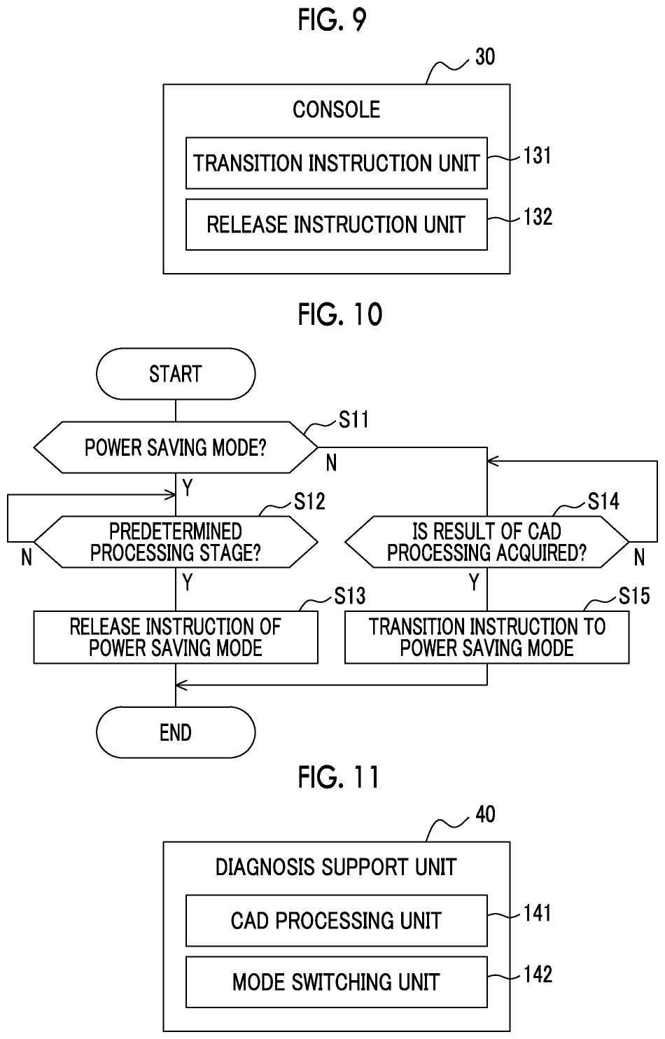

FIG. 9 is a functional block diagram showing an example of the functional configuration of the console 30 in a case where the console 30 performs control related to the suppression of the amount of power consumption in the diagnosis support unit 40. The console 30 includes a transition instruction unit 131 and a release instruction unit 132. The CPU 31 executes the mode switching program 38, whereby the console 30 functions as the transition instruction unit 131 and the release instruction unit 132.

In a case where an operation mode of the diagnosis support unit 40 is not the power saving mode, and in a case where the result of the CAD processing transmitted from the diagnosis support unit 40 is acquired, the transition instruction unit 131 transmits a transition instruction to the power saving mode to the diagnosis support unit 40.

In a case where the operation mode of the diagnosis support unit 40 is the power saving mode, and in a case where a predetermined processing stage among a plurality of processing stages in the medical examination processing shown in FIG. 8 is performed, the release instruction unit 132 transmits a release instruction of the power saving mode to the diagnosis support unit 40.

The release instruction unit 132 may transmit the release instruction of the power saving mode to the diagnosis support unit 40, for example, in a case where the selection screen of the imaging menu is displayed on the touch panel display 34 in Step S1 in the medical examination processing. Alternatively, the release instruction unit 132 may transmit the release instruction of the power saving mode to the diagnosis support unit 40, for example, in a case where determination is made that the irradiation of the radiation is started in Step S2 in the medical examination processing. The release instruction unit 132 may transmit the release instruction of the power saving mode to the diagnosis support unit 40, for example, in a case where determination is made that the irradiation of the radiation is completed in Step S3 in the medical examination processing. The release instruction unit 132 may transmit the release instruction of the power saving mode to the diagnosis support unit 40, for example, in a case where determination is made that the radiographic image is acquired in Step S4 in the medical examination processing. The release instruction unit 132 may transmit the release instruction of the power saving mode to the diagnosis support unit 40, for example, before the transmission of the execution instruction of the CAD processing in Step S5 in the medical examination processing.

The release instruction unit 132 may transmit the release instruction of the power saving mode to the diagnosis support unit 40 in response to an instruction from the user without depending on the processing stage in the medical examination processing. The release instruction unit 132 may transmit the release instruction of the power saving mode to the diagnosis support unit 40, for example, in a case where the execution instruction of the CAD processing is received. The release instruction unit 132 may transmit the release instruction of the power saving mode to the diagnosis support unit 40, for example, in a case where the release instruction of the power saving mode is received. The execution instruction of the CAD processing and the release instruction of the power saving mode can be performed by the user operating the touch panel display 34.

FIG. 10 is a flowchart illustrating an example of a flow of processing that is executed by the CPU 31 of the console 30 executing the mode switching program 38. The mode switching program 38 is executed, for example, accompanied with an execution start of the medical examination processing program 37.

In Step S11, the CPU 31 determines whether or not the operation mode of the diagnosis support unit 40 at the present time is the power saving mode. The CPU 31 transitions the process to Step S12 in a case where determination is made that the operation mode of the diagnosis support unit 40 at the present time is the power saving mode, and transitions the process to Step S14 in a case where determination is made that the operation mode of the diagnosis support unit 40 is not the power saving mode.

In a case where determination is made in Step S11 that the operation mode of the diagnosis support unit 40 is the power saving mode, in Step S12, the CPU 31 determines whether or not a predetermined processing stage among a plurality of processing stages in the medical examination processing shown in FIG. 8 is performed. “A case where the predetermined processing stage is performed” may be, for example, a case where the selection screen of the imaging menu is displayed, a case where the irradiation of the radiation is started, a case where the irradiation of the radiation is completed, a case where the radiographic image is acquired, or a case where the execution instruction of the CAD processing is transmitted, as described above. The CPU 31 transitions the process to Step S13 in a case where determination is made that the predetermined processing stage is performed.

In Step S13, the CPU 31 functions as the release instruction unit 132 and transmits the release instruction of the power saving mode to the diagnosis support unit 40.

On the other hand, in a case where determination is made in Step S11 that the operation mode of the diagnosis support unit 40 is not the power saving mode, in Step S14, the CPU 31 determines whether or not the result of the CAD processing transmitted from the diagnosis support unit 40 is acquired. The CPU 31 transitions the process to Step S15 in a case where determination is made that the result of the CAD processing is acquired.

In Step S15, the CPU 31 functions as the transition instruction unit 131 and transmits the transition instruction to the power saving mode to the diagnosis support unit 40.

FIG. 11 is a functional block diagram showing an example of the functional configuration of the diagnosis support unit 40. The diagnosis support unit 40 includes a CAD processing unit 141 and a mode switching unit 142. The GPU 41 executes the CAD processing program 45, whereby the diagnosis support unit 40 functions as the CAD processing unit 141 and the mode switching unit 142.

The CAD processing unit 141 executes the CAD processing accompanied with the image processing of the radiographic image as a CAD processing target in response to the execution instruction of the CAD processing transmitted from the console 30. Specifically, the CAD processing unit 141 inputs the radiographic image as a CAD processing target to the detection model 46 stored in the nonvolatile memory 43. With this, the detection model 46 detects an abnormal shadow, such as a lesion part, included in the radiographic image as a CAD processing target. The CAD processing unit 141 outputs, for example, positional information indicating a coordinate position in the radiographic image of the abnormal shadow detected by the detection model 46 as the result of the CAD processing. The CAD processing unit 141 may output an image with a mark indicating the position of the abnormal shadow attached to the radiographic image as a CAD processing target, as the result of the CAD processing. The CAD processing unit 141 may specify a type of a disease corresponding to the detected abnormal shadow and may include the specified type in the result of the CAD processing. The CAD processing unit 141 transmits the result of the CAD processing to the console 30.

The mode switching unit 142 switches the operation mode of the diagnosis support unit 40 in response to the transition instruction to the power saving mode and the release instruction of the power saving mode transmitted from the console 30. The mode switching unit 142 transitions the operation mode of the diagnosis support unit 40 to the power saving mode in a case where the transition instruction to the power saving mode transmitted from the console 30 is received. The power saving mode is an operation mode where an amount of power consumption in the diagnosis support unit 40 (GPU 41) is relatively small.

The GPU 41 may operate in synchronization with a clock signal of a relatively long cycle in the power saving mode. Alternatively, the GPU 41 may perform communication with the console 30 (CPU 31) at a relatively low frequency in the power saving mode. The communication may be, for example, communication that is repeatedly performed to inform the console 30 of the presence of the diagnosis support unit 40. The GPU 41 may transition to a sleep state defined in advance, in the power saving mode. In the sleep state, the supply of power is stopped to at least a part of a plurality of circuit blocks configuring the GPU 41. In the power saving mode, the supply of power from the battery 50 to the diagnosis support unit 40 (GPU 41) may be cut off.

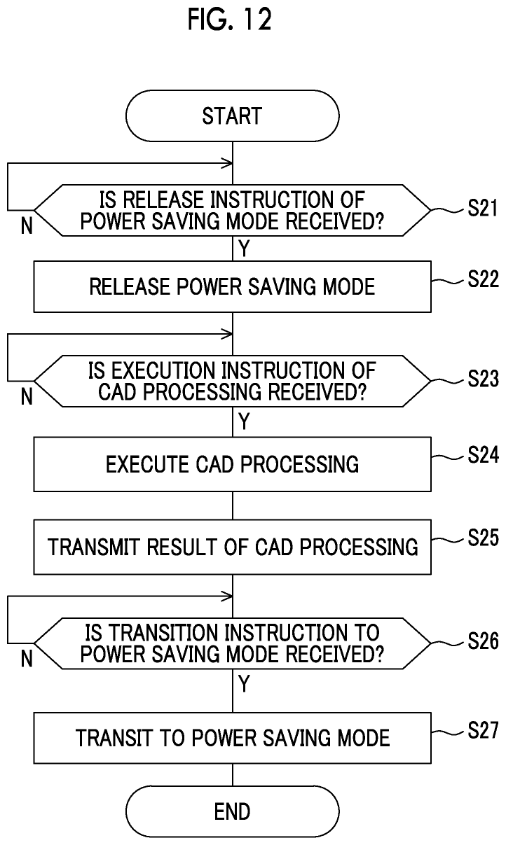

FIG. 12 is a flowchart illustrating an example of a flow of processing that is executed by the GPU 41 of the diagnosis support unit 40 executing the CAD processing program 45. The CAD processing program 45 is executed, for example, accompanied with an execution start of the medical examination processing program 37. It is assumed that, in an initial state, the operation mode of the diagnosis support unit 40 is the power saving mode.

In Step S21, the GPU 41 determines whether or not the release instruction of the power saving mode transmitted from the console 30 is received. The GPU 41 transitions the process to Step S22 in a case where determination is made that the release instruction of the power saving mode is received.

In Step S22, the GPU 41 functions as the mode switching unit 142 and releases the power saving mode. That is, the operation mode of the diagnosis support unit 40 is switched to a normal mode, and a state in which the execution of the CAD processing is possible is brought.

In Step S23, the GPU 41 determines whether or not the execution instruction of the CAD processing transmitted from the console 30 is received. The GPU 41 transitions the process to Step S24 in a case where determination is made that the execution instruction of the CAD processing is received.

In Step S24, the GPU 41 functions as the CAD processing unit 141 and executes the CAD processing accompanied with the image processing on the radiographic image as a CAD processing target transmitted from the console 30 along with the execution instruction of the CAD processing. In Step S25, the GPU 41 transmits the result of the CAD processing to the console 30.

In Step S26, the GPU 41 determines whether or not the transition instruction to the power saving mode transmitted from the console 30 is received. The GPU 41 transitions the process to Step S27 in a case where determination is made that the transition instruction to the power saving mode is received.

In Step S27, the GPU 41 functions as the mode switching unit 142 and transitions the operation mode of the diagnosis support unit 40 to the power saving mode. The GPU 41 may transition to the sleep state in the power saving mode, for example.

As described above, with the medical image processing apparatus 10 according to the embodiment of the technique of the disclosure, after the GPU 41 executes the CAD processing accompanied with the image processing, transition is made to the power saving mode where the amount of power consumption in the GPU 41 is relatively small. With this, it is possible to suppress the amount of power consumption of the GPU 41, compared to a case where the GPU 41 constantly operates in the normal mode. Accordingly, since it is possible to suppress the supply amount of power from the battery 50, it is possible to extend the operation time of the medical image processing apparatus 10. It is also possible to decrease the replacement frequency of the battery 50. With this, it is possible to perform efficient rounds using the medical image processing apparatus 10.

With the medical image processing apparatus 10, in a case where a predetermined processing stage among a plurality of processing stages until the CAD processing is executed in the diagnosis support unit 40 is performed, the power saving mode is released. With this, it is possible to release the power saving mode earlier than a time at which the execution of the CAD processing is possible (for example, a time at which the console 30 acquires the radiographic image). That is, it is possible to start the execution of the CAD processing without delay after the acquisition of the radiographic image.

The CPU 31 of the console 30 may determine a timing of transmitting the release instruction of the power saving mode in consideration of a time (hereinafter, referred to as a return time) needed until the release of the power saving mode is completed after the GPU 41 receives the release instruction of the power saving mode. For example, the CPU 31 of the console 30 may determine the timing of transmitting the release instruction of the power saving mode in consideration of the return time such that a state in which the CAD processing is possible in the GPU 41 is brought until the time at which the radiographic image transmitted from the electronic cassette 60 is acquired. For example, in a case where one minute is needed as the return time, the CPU 31 of the console 30 may transmit the release instruction of the power saving mode to the diagnosis support unit 40 one minute earlier than a time predicted as a time at which the radiographic image is acquired.

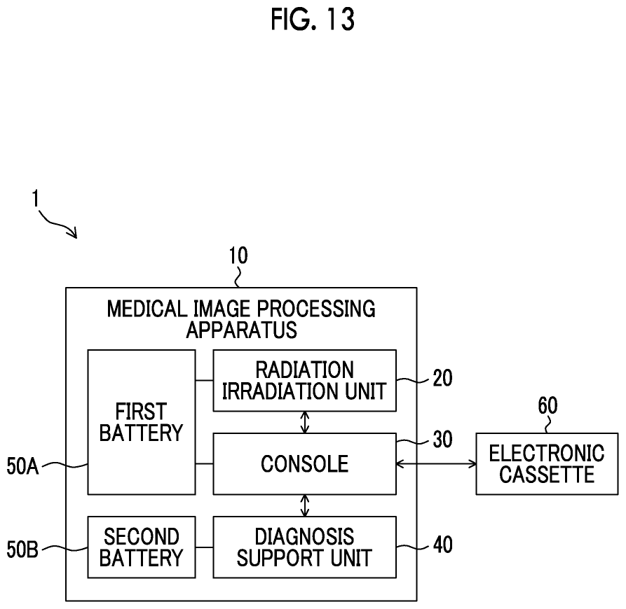

In the embodiment, although a form in which power is supplied to both the console 30 (CPU 31) and the diagnosis support unit (GPU 41) using the single battery 50 has been illustrated, the technique of the disclosure is not limited to the form. For example, as shown in FIG. 13, the medical image processing apparatus 10 may include a first battery 50A that supplies power to the radiation irradiation unit 20 and the console 30 (CPU 31), and a second battery 50B that supplies power to the diagnosis support unit 40 (GPU 41).

In the embodiment, although a case where the diagnosis support unit 40 (GPU 41) transitions to the power saving mode in a case where the transition instruction to the power saving mode transmitted from the console 30 is received has been illustrated, the diagnosis support unit 40 (GPU 41) may transition to the power saving mode without waiting for the transition instruction to the power saving mode after transmitting the result of the CAD processing to the console 30.

In the embodiment, although a case where the radiographic image is applied as the medical image has been illustrated, the medical image may be, for example, an image, such as an ultrasound image or a magnetic resonance imaging (MRI) image, other than the radiographic image.

In the embodiment, although a case where the processing of detecting an abnormal shadow included in the medical image has been illustrated as the CAD processing that is executed by the diagnosis support unit 40 (GPU 41), the technique of the disclosure is not limited to the form. The CAD processing accompanied with the image processing may be, for example, processing of enhancing or attenuating a specific part included in the medical image or may be processing of visualizing change of a specific lesion from past images.

In the above-described embodiment, for example, as the hardware structures of processing units that realizes various kinds of processing, such as the transition instruction unit 131, the release instruction unit 132, the CAD processing unit 141, and the mode switching unit 142, various processors described below can be used. Various processors includes a programmable logic device (PLD) that is a processor capable of changing a circuit configuration after manufacture, such as an FPGA, a dedicated electric circuit that is a processor having a circuit configuration dedicatedly designed for executing specific processing, such as an application specific integrated circuit (ASIC), and the like, in addition to a CPU and a GPU that is a general-purpose processor executing software (program) to function as various processing units, as described above.

One processing unit may be configured of one of various processors described above or may be configured of a combination of two or more processors (for example, a combination of a plurality of FPGAs or a combination of a CPU and an FPGA) of the same type or different types. A plurality of processing units may be configured of one processor.

As an example where a plurality of processing units are configured of one processor, first, as represented by a computer, such as a client or a server, there is a form in which one processor is configured of a combination of one or more CPUs and software, and the processor functions as a plurality of processing units. Second, as represented by System on Chip (SoC) or the like, there is a form in which a processor that realizes all functions of a system including a plurality of processing units into one integrated circuit (IC) chip is used. In this way, various processing units may be configured using one or more processors among various processors described above as a hardware structure.

In addition, as the hardware structure of various processors is, more specifically, an electric circuit (circuitry), in which circuit elements, such as semiconductor elements, are combined can be used.

In the above-described embodiment, although an aspect where the medical examination processing program 37 and the mode switching program 38 are stored in (installed on) the nonvolatile memory 33 in advance, and the CAD processing program 45 is stored in (installed on) the nonvolatile memory 43 in advance has been described, the technique of the disclosure is not limited thereto. Each program described above may be provided in a form of being recorded on a recording medium, such as a compact disc read only memory (CD-ROM), a digital versatile disc read only memory (DVD-ROM), or a universal serial bus (USB) memory. Each program described above may be provided in a form of being downloaded from an external apparatus through a network.

What is claimed is:

1. A medical image processing apparatus comprising:
a first processor;
a second processor that executes image processing on a medical image in response to an instruction from the first processor; and
a battery that supplies power to the first processor and the second processor,
wherein, after the second processor executes the image processing, transition is made to a power saving mode where an amount of power consumption in the second processor is relatively small.

2. The medical image processing apparatus according to claim 1,
wherein the second processor operates in synchronization with a clock signal of a relatively long cycle in the power saving mode.

3. The medical image processing apparatus according to claim 1,
wherein the second processor performs communication with the first processor at a relatively low frequency in the power saving mode.

4. The medical image processing apparatus according to any one of claim 1,
wherein the second processor transitions to a sleep state defined in advance, in the power saving mode.

5. The medical image processing apparatus according to claim 1,
wherein, in the power saving mode, supply of power from the battery to the second processor is cut off.

6. The medical image processing apparatus according to any one of claim 1,
wherein the first processor transmits a release instruction of the power saving mode to the second processor in a case where a predetermined processing stage among a plurality of processing stages until the image processing is executed is performed.

7. The medical image processing apparatus according to any one of claim 1,
wherein the first processor transmits a release instruction of the power saving mode to the second processor in a case where an execution instruction of the image processing is received.

8. The medical image processing apparatus according to any one of claim 1,
wherein the first processor transmits a release instruction of the power saving mode to the second processor in a case where the release instruction of the power saving mode is received.

9. The medical image processing apparatus according to any one of claim 1,
wherein the first processor determines a timing of transmitting a release instruction of the power saving mode such that the second processor is returned to a state in which the image processing is possible, until a time of acquisition of the medical image.

10. The medical image processing apparatus according to any one of claim 1,
wherein the medical image is a radiographic image, and
the medical image processing apparatus further comprises:
a radiation irradiation unit that receives the supply of power from the battery to perform irradiation of radiation for capturing the radiographic image.

11. The medical image processing apparatus according to any one of claim 1,
wherein the second processor outputs information for supporting diagnosis using the medical image through the image processing.

12. The medical image processing apparatus according to any one of claim 1,
wherein a first battery that supplies power to the first processor and a second battery that supplies power to the second processor are provided.

13. The medical image processing apparatus according to any one of claim 1,
wherein the medical image processing apparatus is a mobile type.
